(19) United States
(12) Patent Application Publication
(10) Pub. No.: US 2019/0022166 A1
(43) Pub. Date: Jan. 24, 2019

(54) ORAL ANTI-INFLAMMATORY PEPTIDES TO TREAT EPILEPSY, SEIZURES AND CNS DISORDERS

(71) Applicant: Creative Bio-Peptides Inc., Potomac, MD (US)

(72) Inventor: Michael R. Ruff, Potomac, MD (US)

(73) Assignee: Creative Bio-Peptides Inc., Potomac, MD (US)

(21) Appl. No.: 16/038,144

(22) Filed: Jul. 17, 2018

Related U.S. Application Data
(60) Provisional application No. 62/533,854, filed on Jul. 18, 2017.

(51) Int. Cl. A61K 38/08; A61P 25/08; A61K 38/19; A61P 29/00; A61K 31/70; A61K 38/16

(52) U.S. Cl. CPC A61K 38/08; A61P 25/08; A61K 38/195; A61K 2300/00; A61K 31/70; A61K 38/162; A61P 29/00

(57) ABSTRACT

A method of treating seizures, epilepsy or loss of brain function in an individual comprising the steps of preparing a composition composed of an all-D amino acid peptide and a pharmaceutically acceptable carrier.

The D peptide has the general structure: A-B-C-D-E in which
A is Ser, Thr, Asn, Glu, Ile.
B is Ser, Thr, Asp, Asn,
C is Thr, Ser, Asn, Arg, Lys, Trp,
D is Tyr, and
E is Thr, Ser, Arg, Gly.

And wherein all amino acids in the D peptide are the D stereoisomeric configuration and said peptide composition is administered in a therapeutically effective dose wherein said composition acts to suppress inflammation underlying the loss of brain function. The D peptide may be esterified, glycosylated, or amidated at E to enhance tissue distribution by promoting egress from the circulation and penetration into the brain.

This application claims the benefit of U.S. Provisional Application Ser. No. 62/533,854, filed Jul. 18, 2017.

### FIELD OF THE INVENTION

The present invention relates broadly to the treatment or prevention of epilepsy or seizures, spontaneous or induced, that might ensue after an episode of status epilepticus, post traumatic epilepsy (PTE), or as a complication of head trauma such as mild, moderate, or severe traumatic brain injury, intracranial hemorrhage due to concussions, skull fracture, traumatic encephalopathy, concussive blasts and neurodegeneration, including those caused by neurosurgical procedures as well as by brain injuring events in general, including organophosphate (OP) nerve agent exposure, brain infections (bacterial, viral, parasitic),

encephalitis, toxic shock, eclampsia, intracranial hemorrhage, cerebral palsy, hypoxia, hyponatremia, drug overdose, Alzheimer's Disease, brain tumors, stroke, autism spectrum disorders, congenital conditions like Down's syndrome, Angleman's syndrome, tuberous sclerosis, neurofibromatosis, or genetic forms with ill-defined cause.

### BRIEF DESCRIPTION OF THE DRAWINGS

FIG. 1A-D illustrate that all-D-TTNYT (SEQ ID NO:1) blocks TLR4-mediated maturation of antigen presenting dendritic cells.

FIG. 2 Illustrates all-D-TTNYT (SEQ ID NO:1) potently blocking both MCP-1(CCL2) and MIP-1β(CCL4)-elicited chemotaxis of human monocytes at CCR2 and CCR5 receptors, respectively.

FIG. 3 Illustrates the effects of three additional all-D amino acid peptides in blocking CCL2 (MCP-1) chemotaxis at low concentration.

FIG. 4A-F Illustrate all-D-pentapeptide TTNYT (SEQ ID NO:1) lowering expression of chemokines CCL2 and CCL3, chemokine receptors CCR2 and CCR5, and cytokines IL-1 and TNF [symbol] in rats.

### INTRODUCTION

Epilepsy is a neurological disorder of the brain that predisposes a person to recurrent unprovoked seizures as a result of uncontrolled electrical activity in the brain. Each year, about 150,000 Americans are diagnosed with this central nervous system disorder. Over a lifetime, one in 26 people will be diagnosed with epilepsy. There are many causes of epilepsy and not all seizures are considered to be epilepsy as there are numerous other brain disturbances and injuries that promote seizure activity. The four most common causes of seizures are head trauma, stroke, brain tumor, and brain infection. Other causes include nerve agent toxicity, drug effects or intoxication, genetics, and metabolic disturbances.

TBI is an etiological factor in up to 20% of epilepsies in the general population. Post-traumatic seizures (PTS) and post-traumatic epilepsy (PTE) are complications from traumatic brain injury (TBI) which significantly worsen functional outcome. It is estimated that 1.7 Million Americans sustain a traumatic brain injury (TBI) each year, ranging from mild to severe, and, in the U.S., this is in addition to about 360,000 soldiers involved in combat operations and public safety workers surviving terrorist attacks who develop TBI secondary to explosive (concussive) blasts. The risk of epilepsy after traumatic brain injury ranges from 1.5 for mild injury to 17.2 after severe injuries involving subdural hematoma, skull fracture, loss of consciousness or amnesia of 1 day or more, and age over 65 years. Patients with penetrating head injuries carry a high risk of developing PTE decades after their injury, and 80,000 to 90,000 Americans experience long-term disability from TBIs.

Infections of the CNS are also a major risk factor for epilepsy. A wide variety of CNS infections, including bacterial (meningitis, tuberculosis), viral (e.g. herpes simplex, HHV-6, HIV), parasitoses (e.g. cerebral toxoplasmosis, malaria), fungal (e.g. candidiasis, coccidioidomycosis, aspergillosis), and prion infections, can lead to status epilepticus. Thirty to fifty percent of individuals with HSV-1 encephalitis develop seizures, and viruses within the Picornaviridae Family, such as enteroviruses, Coxsackieviruses A and B, parechoviruses and echoviruses have been associated with the development of febrile seizures. Enteroviruses are a leading cause of viral encephalitis in children, which can cause severe seizures. CNS infections are associated with brain inflammation, and brain inflammation from any cause is a seizure risk.

Exposure to organophosphate (OP) nerve agents (NA) also creates a state of brain injury and inflammation that induces a condition of status epilepticus (SE) which sets off a brain damaging cascade of seizures which may result in death. SE is characterized by multiple or prolonged seizures with a rapid and sustained neuroinflammatory response marked by activation of microglia and astrocytes and significantly increased brain levels of proinflammatory mediators, including chemokines and cytokines (1, 2). Neuroinflammation plays a key role in the pathogenesis of OP-nerve agents, which often results in permanent brain damage.

In non-lethal exposures status epilepticus (SE) can cause seizure-related brain damage, leading to persistent cognitive and behavioral deficits, including depression, and sleep-wake disturbances, sensorimotor-related comorbidities, accompanied by reduced neurogenesis.

Current post-exposure treatments for nerve agent-induced exposure (atropine, oxime, and high-dose benzodiazepines) is insufficient and is effective only if administered within minutes of exposure. Survivors, even with treatment, often suffer from long-lasting adverse effects, including mild-to-severe decline in memory and behavioral changes, affective disorders, and recurrent seizures There are no agents to treat the neurodegenerative sequelae of NA exposure, and there still remains a significant treatment need for survivors of acute nerve agent intoxication with highly refractory, recurrent, or diazepam resistant seizures.

Epilepsy refers to many types of recurrent seizures produced by paroxysmal excessive neuronal discharges in the brain; the two main generalized seizures are petit mal, which is associated with myoclonic jerks, akinetic seizures, transient loss of consciousness, but without convulsion; and grand mal which manifests in a continuous series of seizures and convulsions with loss of consciousness. Anything causing a structural or functional derangement of brain physiology may lead to seizures, and recurrent seizure events may be labelled “epilepsy.”

The mainstay of treatment for seizure disorders has been the long-term and consistent administration of anticonvulsant drugs, and today many such drugs are well known. Several anti-epileptic drugs, such as phenytoin, phenobarbital, carbamazepine and valproic acid, are effective for the prevention of early PTS, but not late PTS or PTE. Unfortunately, despite the many available pharmacotherapeutic agents, a significant percentage of the population with epilepsy or related disorders are poorly managed. Moreover, none of the drugs presently available are capable of achieving total seizure control, do not treat the underlying cause of seizure activity which is both acute and persistent inflammation, and most have unfavorable side effects which limit their use.

### DETAILED DESCRIPTION

The present invention relates to compositions for treatment or prevention of epilepsy or seizures and a method for modulating, in particular reducing, an excessive immune response in an animal, such as a human or another mammal, specifically in the brain due to injury, trauma or infection resulting in activation of innate immune inflammatory pathways which cause excessive cytokine, chemokine, and TLR4/MyD88 receptor activation which leads to seizures, loss of normal function, loss of neurons, cognitive impairment, and even death.

Accumulating data suggests that the adaptive as well as innate immune system pathways are directly involved in the pathogenic mechanism(s) of epileptogenesis (3) and that inflammation, in turn, influences the occurrence and severity of seizures, and seizure-related neuronal death. In support of the role of innate immune inflammatory reactions as a causative etiology in the epileptic, neurodegenerative, and cognitive pathologies of seizure activity, elevated IL-1, TNFα and IL-6 have been detected in sera from people with temporal lobe epilepsy (TLE) (4) and from resected brain tissue from people with intractable epilepsy (5). In animals exposed to OP agents there was a significant increase in IL-1 [symbol] , TNF [symbol] , IL-6, prostaglandin E2, and chemokines (CCL2, CCL3, CCL5) in the cortex and hippocampus, which remain elevated for days (6).

A hippocampal infusion of lipopolysaccharide (LPS) induced epileptic seizures in rats with enhanced expression of IL1β, TNFα, and neuronal nitric oxide synthase (7) indicating that the TLR4/MyD88 innate immune pathway is an inducer of acute seizure activity. Even systemic exposure to the inflammagen LPS in an early post-natal period in mice caused brain activation of astrocytes, microglia, and promoted excitatory synapse development, leading to enhanced seizure susceptibility in a MyD88-dependent manner (8). Thus, an early developmental inflammatory insult can cause a life-long predisposition to seizure generation. Deleting MyD88 or suppressing Erk1/2 in astrocytes rescued LPS-induced developmental abnormalities suggesting these pathways can be effective targets for anti-seizure drug interventions.

Peripheral inflammatory stimuli can also impact on seizure propensity and chemokines may play a role in leukocyte migration into brain during the neuroinflammatory processes of seizures and epilepsy described above. The chemokine MCP-1 (CCL2) is one of the most elevated inflammatory mediators in patients with pharmacoresistent epilepsy, and CCL2 has a pro-convulsant effect (9). Intracerebral administration of anti-CCL2 antibodies abrogated LPS-mediated seizure enhancement in chronically epileptic animals. These results show that CCL2 is a key mediator in the molecular pathways that links peripheral inflammation with neuronal hyperexcitability and demonstrate a crucial role for CCL2 and its receptor CCR2 in seizure control.

Cortical contusions due to head injuries cause release of excitatory neurotransmitters (glutamate, acetylcholine, and aspartate) which generates free radicals and excitotoxicity that can kill neurons and cause seizures. The glutamate analog kainic acid (KA) is used experimentally to cause a pattern of seizure-related brain damage in rats that closely resembles that observed in human epilepsy.

An experimental rat model based on intraperitoneal KA administration showed that induced seizures elevated the expression of the CCR5 ligands MIP-1α (CCL3) and RANTES (CCL5) in the microvasculature and increased brain infiltration of CCR5+ cells, effects associated with neuronal loss, inflammation, and gliosis in the hippocampus (10). CCL5 facilitated release from mouse synaptosomes of the excitatory neurotransmitter aspartate, a pro-seizure effect, which was blocked by DAPTA (11), a CCR2/CCR5 dual-receptor antagonist (12). Decreased CCR5 strongly protected from excitotoxin-induced seizures, BBB leakage, CNS injury, inflammation, and facilitated neurogenic repair (10).

IL-1b, IL-6, and TNFα are known to be among the major cytokines up-regulated during the acute-phase response to nerve-agent toxicity, CNS stress, and injury. The release of TNFα and other inflammatory cytokines exacerbates the activation of glial cells and the physiological response switching the Th1 to Th2 cycle and promoting gliosis, inhibiting astrocytic glutamate uptake and inducing apoptosis, particularly in oligodendrocytes thereby contributing to damaging demyelination. The ability to inhibit the action of TNFα and other inflammatory activators in the early phases of traumatic CNS injury may yield a salutary clinical outcome to prevent or limit seizure risk.

Further support for these receptor targets in seizures associated with brain injury comes from reports that blocking CCR2/CCR5 is protective in stroke (13, 14) and traumatic brain injury (TBI) (15), where a dual CCR2/5 antagonist, significantly ameliorated injury-induced sequelae in the aged TBI animals

These findings indicate that treating the underlying innate immune inflammation, by blocking cytokine production, chemokine release, activation of MyD88 pathways, suppression of microglial and astrocyte activation, infiltration of peripheral monocytes into brain, and prevention of chemokine mediated excitatory amino acid release will provide a constellation of unique therapeutic effects to yield novel treatment opportunities in seizures and epileptogenic conditions of diverse origin (16).

DAPTA and peptides of the subject invention block the chemokine receptors CCR2 andCCR5 and shift the cytokine balance from inflammation (M1) to repair (M2) responses(12, 17, 18), in part by blocking transcription factors NFkB (19) and STAT3 (20). DAPTA specifically reduced the cytokines TNFα, IL-1, 6, and 8, and elevated IL-4, 10, and 13 in five tested individuals with HIV(18) (Table 1). Reduced TNFα, IL-1, 6, and 8 and shift to IL-4, IL-10-responses are neuroprotective and survival enhancing in TBI (21, 22).

DAPTA, and the related analogs of this invention have a useful and novel action in that they are not soley “antagonists of inflammation”, but rather they have a more nuanced and effect to shift the cytokine profile from an “M1” state, to an “M2” state (Table 1), which is a uniquely beneficial therapeutic effect in neurodegenerative conditions (23). Such effects would be expected to have benefits in brain injury and anti-seizure effects in people. More broadly, by virtue of shifting the cytokine profile (M1 to M2) of the brain microglia that play a fundamental role in neurodegenerative diseases such as Alzheimer's disease (AD), Parkinson's disease (PD), and multiple sclerosis (MS) (23), the peptides of this invention will provide treatment benefits in these and other neurodegenerative conditions.

In particular embodiments, the invention relates to the prevention or treatment of damage to neurons, loss of neurons, and neuronal hyperexcitability associated with chronic immune activation occurring after a brain injury that occurs via cytokine, chemokine, and toll-receptor/MyD88 inflammatory pathways. A specific embodiment is a therapeutic composition comprising an anticonvulsant effective amount of a peptide according to the present invention.

The present invention also relates to compounds, compositions and methods for the treatment of conditions associated with enhancement or improvement of cognitive ability or to counteract cognitive decline so that it more closely resembles the function of an aged-matched normal, unimpaired subject. In one embodiment of the invention, the person has normal cognitive function which is enhanced. In a further embodiment, the person exhibits cognitive impairment associated with brain injury due to neuroinflammation, such as occurs in aging, which is improved.

In still a further embodiment a person with cognitive impairment associated with a disease or disorder such as Alzheimer's disease, mild cognitive impairment (MCI), autism, dyslexia, attention deficit hyperactivity disorder, compulsive disorders, psychosis, bipolar disorders, depression, Tourette's syndrome and disorders of learning in children, adolescents and adults, Age Associated Memory Impairment, Age Associated Cognitive Decline, Parkinson's Disease, Down's Syndrome, traumatic brain injury, neuro-AIDS, Huntington's Disease, Progressive Supranuclear Palsy (PSP), HIV, stroke, vascular diseases, Pick's or Creutzfeldt-Jacob diseases, multiple sclerosis (MS), other white matter disorders, schizophrenia, and drug-induced cognitive worsening may be improved.

In pre-clinical animal models, DAPTA and the peptides according to the present invention prevented loss of neurons with aging and blocked activated microglial mediated neurotoxicity and release of cytokines and chemokines associated with cognitive decline. In humans DAPTA improved cognition, speed of information processing and functional brain imaging in neuro-AIDS (24, 25). Therefore, it is believed that the compounds according to the present invention have a strong potential to improve cognitive deficits in the indicated conditions due to shared neurodegenerative inflammatory mechanisms.

Dyskinesias are a group of disorders often involving the basal ganglia in which unwanted, superfluous movements occur. Defects in the basal ganglia may results in brisk, jerky, purposeless movements that resemble fragments of voluntary movements. Dyskinesias may include any combination of involuntary, rapid, randomly irregular jerky movements (chorea); relatively slow, writhing motions that appear to flow into one another (athetosis); increased muscle tone with repetitive, twisting, patterned movements and distorted posturing (dystonia); and uncontrollable flinging movements of an arm, a leg, or both (ballismus).

Primary dyskinesias occur in a number of different diseases. Sydenham's chorea is a disease usually associated with a toxic or infectious disorder that apparently causes temporary dysfunction of the corpus striatum and usually affects children. Huntington's chorea (HD) is a dominant hereditary disorder that begins in middle life and causes mental deterioration and progressive degeneration of the corpus striatum in affected individuals. Cerebral Palsy is a general term referring to defects on motor functions or coordination resulting from several types of brain damage, which may be caused by abnormal brain development or birth-related injury. Some symptoms of cerebral Palsy such as athetosis are related to basal ganglia dysfunction.

Secondary dyskinesias are observed in various diseases either as a secondary symptom (head injury, multiple sclerosis) or as a consequence of drug treatments. Parkinson's disease (PD), characterized by muscular rigidity, loss of facial expression, tremor, a slow, shuffling gait, and general lack of movement, is caused by a dysfunction in the substantia nigra. The increased muscular rigidity in Parkinson's disease results from defective inhibitions of some of the basal ganglia by the substantia nigra. The most common types of dyskinesias are chorea and dystonia, and these are often mixed. Studies on Huntington's Disease demonstrated an altered immune response with activated microglia and secretion of IFNα, IL-10, IL-8 and IL-1β(26), identifying specific immuno-pathological targets in dyskinesias which may be normalized with the subject peptides.

In still a further embodiment a person suffering from headache may benefit as the pain may have an inflammatory cause (inflammatory headaches). The most common type of vascular headache is migraine. After migraine, the most common type of vascular headache is headache produced by fever. Pneumonia, measles, mumps, and tonsillitis are among the diseases that can cause severe toxic vascular headaches. Toxic headaches can also result from the presence of foreign chemicals in the body. Other kinds of vascular headaches include “clusters,” which cause repeated episodes of intense pain, and headaches resulting from a rise in blood pressure. It is believed that the compounds according to the present invention have a strong potential to improve headaches in the indicated conditions due to the efficacy of epilepsy medications in these conditions, coupled with more recent studies that show inflammatory incitement of seizure propensity.

The present invention is also directed to the use of a class of peptide compounds for treating diseases associated with hyperexcitability, such as diseases associated with a hyperexcitable tissue and which may be associated with dysfunction of an ion channel, such as a glutamate-NMDA receptor. Hyperexcitability is defined as an abnormal increase in responsiveness of a central or peripheral nervous system neuron to synaptic input, and may be caused by a pathophysiological inflammatory event. Examples of diseases associated with hyperexcitability are channelopathies, dystonia, myotonias, myasthenias, ataxias, long QT syndromes and anxiety- and stress-diseases.

The mode of action of the present compounds differs from that of common drugs used for the treatment of hyperexcitability that typically affect ion channels to affect signal propagation in excitable tissues. In contrast, peptides of the present invention block the action of chemokines that facilitate release of excitatory neurotransmitters (27) that facilitate epileptogenesis.

Some examples of DAPTA anti-inflammatory benefits related to the present invention supported in animal models include traumatic brain injury (28), neuropathies of diverse origin (29, 30), including diabetic neuropathies, blocking the release of neurotoxic excitotoxins (11) and in recovery from stroke/cerebral ischaemia (14). Use of DAPTA or the subject all-D-peptides may be beneficial treatments in these and further conditions due to common inflammatory mechanisms with epileptogenic activity after brain injury.

The embodiments disclosed teach a general method of how to make small receptor-active peptides of five to twenty amino acids that alleviate the inflammatory response by shifting the cytokine balance in a number of conditions which lead to increased seizure and epilepsy risk. These include: brain injury, nerve agent exposure, Alzheimer's Disease, intracrainial hemorrhage, brain tumors, stroke, autism, congenital conditions like Down's syndrome, Angleman's syndrome, tuberous sclerosis, neurofibromatosis, or genetic forms with ill-defined cause, viral, bacterial, fungal or other infections; and in particular, any disease wherein infection can manifest in an opportunistic fashion, e.g. during antiviral or immunosuppressive therapy or in any situation where an immunosuppressed state exists, or in immune reconstitution inflammatory syndromes.

### Method to Create Oral Bioavailibility

Practitioners skilled in the art of peptide design understand that it is overwhelmingly the case that modifications of the peptide backbone, including substitution of D-amino acids, particularly at receptor-active sites in the peptide, cause loss of activity, and in some modifications complete inactivity. In fact, the use of D-amino acid substitutions is commonly used to identify, by loss of function, critical pharmacophore residues in a peptide.

Thus, an unexpected and non-obvious aspect of the present invention is the use of all-D amino-acids in the creation of the orally bioactive peptides that target chiral molecules, such as cell surface GPCR receptors. A recent review (31) of oral delivery of therapeutic proteins and peptides indicates that “Despite extensive research efforts, oral delivery of a therapeutic peptide or protein is still a challenge for pharmaceutical industries and researchers. Therefore, because of the short circulatory half-life exhibited by peptides in vivo, they need to be administered frequently resulting in increased cost of treatment and low patient compliance” and in many cases oral delivery is not even possible. Generally, protein and peptide drugs are rapidly denatured or degraded by the low pH environment of the gastric media or the hydrolytic enzymes in the gastrointestinal tract and oral dosing is a preferred route that is typically difficult to achieve with receptor-targeting peptides.

Chiral selectivity of ligand action at receptors is not surprising and is well understood as a principal of enzymology. For example, a chiral specificity is noted in majority of the NSAIDs (non-steroidal anti-inflammatory drugs). For NSAIDs the enantiomer with S configuration almost exclusively possesses the ability to inhibit prostaglandin activity. R-enantiomers of NSAIDs have poor COX inhibitory activity (32). The opiate receptor is an example of a G-protein coupled receptor showing ligand stereoselctivity, in which levorphanol is the active analgesic component of the racemic mixture racemorphan, while its stereoisomer dextrorphan, is inactive.

Some examples of all-D-peptide activity exist, such as the anti-microbial human θ-defensins, which are cationic peptides which disrupt bacterial, but not mammalian, cell membranes. There is no stereo-selective biological interaction of a cationic peptide to a membrane. Defensin activity is derived from a charge disruption of a membrane. This is different from the action of the present inventive peptides which target stereo-specific cell surface receptors and are highly sensitive to ligand conformation and shape.

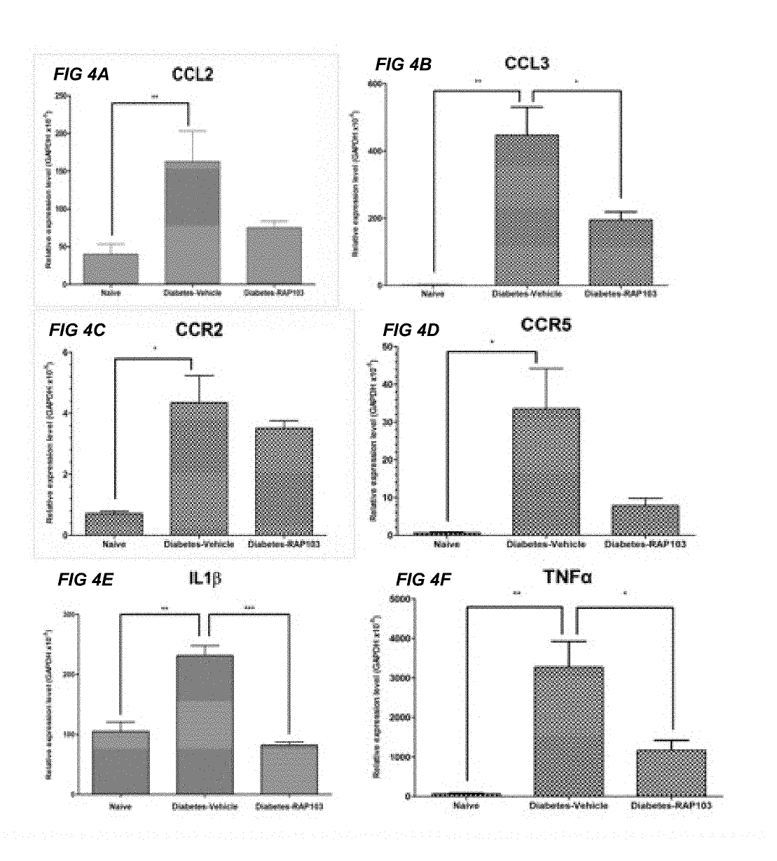

The bioactivity of a receptor active all-D peptide is an unexpected and non-obvious aspect of the present invention in view of an earlier study, Pert (33), FIGS. 3 and 4, and the related U.S. Pat. No. 5,276,016 which showed that that D for L substitutions in linear peptide ASTTTNYT (SEQ ID NO:14) can cause great loss of potency.

Having one D substitution in DAPTA, in the specific position No1, (the D-ala) retains receptor potency, primarily as this residue of the peptide is not needed for bioactivity, indeed may be completely removed. The terminal pentapeptide however is responsible for the biopotency, and D amino acid modifications of these residues are not well tolerated.

Thus, making an additional D substitution in DAPTA, in the terminal pentapeptide required for activity, at position No 8 (the D-Thr), results in loss of 99 to 99.9% of the activity. It is therefore shown that introduction of L to D substitutions cannot be made in a general fashion, and that these modifications can, and typically do, destroy biopotency by disrupting the peptide structure required for receptor potency.

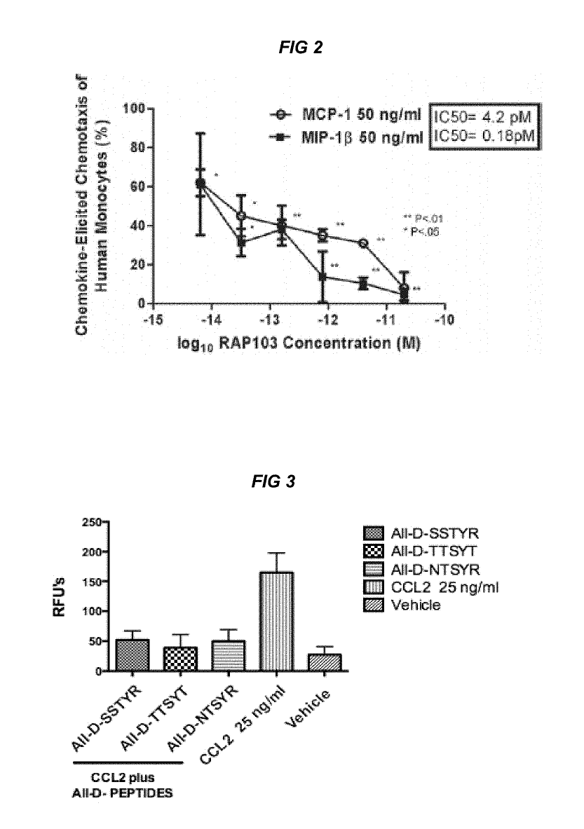

This point is further made in Brenneman, 1988 (34), with specific reference to the peptide TTNYT (SEQ ID NO:1). See FIG. 2 and Table 1. Upon making the L to D substitution in position 4 (Tyr), the peptide completely loses activity.

A detailed study of the peptide TTNYT (SEQ ID NO:1) and L to D substitutions was published in

Smith, 1988 (35), Refer to FIG. 3. Introduction of single L to D substitutions in each position 1,2,3,4, results in loss of potency, and all of the D-amino acid substitutions are substantially less active (50×) to completely inactive.

The notion that an all-D peptide would retain significant potency is furthermore novel in consideration of long accepted art of Stewart and Woolley (36) who prepared all-D peptides of a hormone. For example, from their article, “In contrast to the change of a single residue, the inversion of all the amino-acid residues in a pentapeptide which has hormonal activity of MSH was found to cause loss of hormonal activity . . . ”

Further in this paper the authors write “because there is as yet no general method for predicting the structural requirements required to make antimetabolites of peptides, we synthesized all-D bradykinin (note 9 amino acids, similar size to the 8-amino acid Formula 1 peptide of Andersen) in an effort to find out whether inversion of all the amino-acids of a peptide may be a generally applicable method for synthesis of peptide antagonists.”

The authors then concluded: “Amounts of all-D-bradykinin up to 50,000 times the the standard challenge of bradykinin showed neither any inhibition of the response to bradykinin, or any bradykinin-like effect. It would thus seem that inversion of all the amino-acid residues may not be a generally applicable method for formation of antimetabolites of biologically active peptides”.

Michaelis and Trigg (U.S. Pat. No. 5,798,335) have claimed modified analogs of DAPTA that incorporated D-amino acids in some, but not all, positions. Andersen et al (U.S. Pat. No. 6,011,014 and U.S. Pat. No. 6,265,374) also claim a treatment of inflammation and multiple sclerosis using DAPTA and modified analogs of DAPTA that incorporated D-amino acids in some, but not all, positions. No reduction to practice for any all-D-amino acid modified peptide was proposed or provided, and no example of claimed benefit or treatment use with an all-D-amino acid pentapeptide was illustrated. No all-D-peptide of SEQ ID NO: 1-13 of the present invention was claimed in these prior applications.

The ability to make D for L amino acid substitutions in all positions however creates the possibility to make orally stable peptide compounds. Stability of peptides in target tissues due to digestive enzymes has limited their broad utility. The ability to create all-D peptides that retain potency is an unexpected general method of creating peptides SEQ ID NO: 1-13, and possibly others, which may be stabilized to proteolysis, while retaining biopotency, so these peptides benefit from enhanced stability.

Thus, neither Pert et al. (U.S. Pat. No. 5,276,016), who first used D-amino acids in the octapeptide Peptide T (ASTTTNYT) (SEQ ID NO:14) to create the analog DAPTA (Dala1-peptide T-amide), or Michaelis and Trigg (U.S. Pat. No. 5,798,335), or Andersen et al (U.S. Pat. No. 6,011,014 and U.S. Pat. No. 6,265,374) teach substitution of all of the naturally occurring L-amino acids by D-amino acids in Peptide T or DAPTA.

The use of D-substitutions in “each” position claimed by Michaelis and Trigg or Andersen et al., cannot be inferred to mean in “all” positions, and in any event, has not been reduced to practice in these inventions. The data of Brennemen, 1998 (34) and Smith, 1988 (35) shows that in no instance does a D for L amino-acid substitution in Sequence ID 1 achieve comparable potency to the all-L form, rather D substitutions result in loss of activity, sometimes complete loss of biopotency in a position dependent fashion.

Therefore, it cannot be claimed that making all of the amino acids into D-form is obvious. The specific facts relating to the peptides of this invention from the prior published art inform the exact opposite view, that making an all-D peptide would not be efficacious as an anti-inflammatory agent that targets innate immune system G-protein coupled receptors, such as the chemokine receptors.

This type of structure-function analysis is a key to drug design and must be determined experimentally in each instance. Our recognition that a pentapeptide fragment of DAPTA (Sequence ID 1) comprised of all-D-amino acids retained substantial potency led us to determine that other peptapeptides retained activity as all-D-amino acid forms in specific chemokine receptor functional tests.

The use of all-D-amino acids containing peptides related to SEQ ID NO: 1 that retained substantial biopotency to block CCR5 receptors was first disclosed in U.S. Ser. No. 12/688,862, however no oral use was enabled or claimed, nor have any prior disclosures including Appl. No.: U.S. Ser. No. 13/024324 identified uses to treat epilepsy, seizures, dyskinesia's, or headaches resulting from brain injury, NA exposure, or from any cause.

A peptide of the present invention (all-D-TTNYT) (SEQ ID NO:1) has previously been proposed to be effective in modulating inflammation caused by CCR5 receptors (U.S. application Ser. No. 12/688,862, US 2010/0184705 A1). A further use in reducing pain in peripheral neuropathy (Ser. No. 13/024324), by targeting CCR5, CCR2 and CX3CR1 chemokine receptors, has been disclosed. Neither of these applications teaches a use in preventing seizures, epilepsy, dyskinesia's, or headaches. A prior patent U.S. Pat. No. 5,248,667 teaches a method of treating psoriasis by use of the peptide “DAPTA” and related D-peptides, but not peptides of all-D composition.

Oral delivery solves another problem that is common with peptides, their propensity to aggregate in liquid solutions and lose biopotency, as the peptides may be compounded in solid forms, such as oral pills, with long shelf lives.

### Method to Enhance Stability of Liquid Solutions

In some instances, a liquid formulation may be desired, such as use by injection in an unconscious person. In that case a means of limiting peptide aggregation in solutions must be employed. Such an improvement can be accomplished by addition of sugar monomers of the aldohexose series of carbohydrates, an example is D-mannose, and an effective concentration is 20 mg/ml, although other concentrations are effective. Additional resistance to aggregation can be achieved by addition of an aromatic alcohol. The benzyl-group interacts with the tyrosine moiety of the subject peptides to prevent their “stacking” with each other and prevent aggregation of the peptide solution. An example aromatic alcohol is benzyl alcohol, which is often used as a bacteriostatic preservative in intravenous medications. Its use here is to prevent peptide aggregation. Concentrations of 0.5% benzyl alcohol are useful, although other concentrations are effective. The combination of mannose and benzyl alcohol is particularly efficacious as a preferred embodiment to stabilize aqueous pharmaceutical compositions of the subject peptides and prevent their aggregation upon storage of liquid solutions. Such improvement permits emergency rescue use of the peptides by parenteral administration in persons not able to ingest an oral pill, or use as a liquid nasal spray.

### Method to Enhance Entry Into Brain

Water soluble peptides, such as those of the present invention, are normally not transported through the brain capillary wall, i.e. the blood-brain barrier (BBB). Chimeric peptides may be transportable through the BBB and are formed by the covalent coupling of a nontransportable peptide, e.g. β-endorphin, to a transportable peptide vector, e.g. cationized albumin. A simpler approach for peptides of the present invention is to “cationize” the peptide directly by neutralizing the charge of the terminal COOH moiety at physiological pH. Thus, modifications of the subject peptides such as esterification, glycosylation, or amidation can be made to enhance their tissue distribution, specifically entry into the brain by charge cationization of the peptide at physiological pH in the range of 6 to 8. Previously the terminal amide modification was introduced by Pert et al. (U.S. Pat. No. 5,276,016) to provide protection from carboxypeptidase degradation of DAPTA, and others, including Michaelis and Trigg or Andersen et al., who also have employed this rationale. That is not the function here, as Sequence ID 1 is fully protected to degradation and needs no terminal amide (—NH2), ester, or glycosyl moiety to block proteases and confer resistance to degradation. None of the prior art related to amidated peptides of the present invention claimed or disclosed any improvement in tissue distribution or entry into brain. A further novel property of this invention concerns increased tissue distribution and entry into brain of “cationized” peptides which is achieved by esterification, glycosylation, or amidation. Therefore, such modification provides an additional and novel improvement to the specific peptides of this invention by enhancing their egress from the circulation and delivery to target issues.

### Method to Enhance Peptide Half-life and Bioavailibility

The rapid renal clearance of peptides in vivo limits the treatment of diseases that require constant receptor targeting, which might be achieved with longer peptide half-lives. One approach for extending peptide circulation times is accomplished by binding a peptide ligand to the long-lived serum protein albumin. Acylation with fatty acids is the most successful strategy for delaying peptide clearance. The attachment of either myristic or palmitic acid to insulin and GLP-1 led to the creation of daily use acylated peptide drugs. Fatty acids combined with a short linear peptide has been used to create a peptide-fatty acid chimer able to bind albumin with increased affinity and has extended the elimination half-life approximately 25-fold (Zorzi et al., 2017).

In accordance with this strategy, we propose that peptides of the present invention may be modified by acylation, such as by attachment of myristic or palmitic acid, in some instances by addition of an N-terminal glycine to a peptide. An example is a peptide of the form Myr-GTTNYT (SEQ ID NO:15), or Myr-GTTNYT-NH₂ (SEQ ID NO:15). Such a ligand would have a prolonged elimination half-life, and enhanced tissue delivery. Use of a fatty acid combined with a short linear peptide ‘tag’, as in Zorzi et al., would have an even longer elimination half-life.

By the multiple combined improvements disclosed in this invention, specifically: 1) a treatment for seizures, epilepsy, dyskinesia's, or headaches, 2) achieving oral bioavailability by use of the all-D amino acid modifications that unexpectedly retain receptor biopotency, 3) reduced size compared to DAPTA (pentapeptide compared to an octapeptide) to simplify manufacture and cost, and in some uses 4) “cationization” of the peptide so that the C-terminal carboxcylic acid may be esterified, glycosylated, or amidated to further enhance tissue distribution, and 5) acylation, to extend therapeutic half-life, a peptide may be administered to individuals seeking modification of excessive inflammation such as in epilepsy, seizures, and brain injury, the subject invention creates an efficacious composition that provides the desired and novel treatment benefits.

### Other Active Compounds

Applicant believes other pentapeptides comprised of all-D-amino acids will be effective, including the peptides: SSTYR, STNYT, TTSYT, NTSYG, ETWYS, NTSYR, INNYT, IDNYT, TDNYT, TDSYS, TNSYR and NTRYR, (SEQ ID NOS 2-13, respectively) as well as the octapeptide ASTTTNYT (SEQ ID NO:14).

According to a first aspect of the present invention, there is provided the use of a linear peptide of SEQ ID NO: 1 wherein all amino acids are in the D-stereoisomeric configuration:

Sequence ID 1: A-B-C-D-E wherein:

A is Ser, Thr, Asn, Glu, Arg, Ile, Leu,

B is Ser, Thr, Asp, Asn,

C is Thr, Ser, Asn, Arg, Gln, Lys, Trp,

D is Tyr, and

E His Thr, Ser, Arg, Gly.

Candidates for E may be esterified, glycosylated, or amidated.

The peptides or peptide formulations may be used alone or in combination with any other pharmaceutically active compound or an excipient to treat the inflammation of epilepsy, seizures, and brain injury. Useful pharmaceutical compositions may comprise a peptide of this invention and at least one further compound for the prevention, alleviation or/and treatment of seizures wherein the effect of this composition in the prevention, alleviation or/and treatment of seizures is synergistic as compared to the effect of the respective compounds given alone. Examples of combination compositions would be a peptide with a racetam, lacosamide, dibenzazepine, sulfamate, phenytoin, or barbiturate.

The peptides may be administered orally, bucally, parenterally, topically, rectally, vaginally, by intranasal inhalation spray, by intrapulmonary inhalation or in other ways. In particular, the peptides according to the invention may be formulated as pills for oral administration, in controlled release formulations, for injection (for example subcutaneous, intramuscular, intravenous, intra-articular or intra-cisternal injection), for infusion, and may be presented in unit dose form in ampoules or tablets or in multidose vials or other containers with an added preservative. The compositions may take such forms as suspensions, solutions, or emulsions or gels, and may contain formulatory agents such as suspending, stabilizing and/or dispersing agents. Alternatively, the active ingredient may be in powder and/or lyophilized form for direct administration or for constitution with a suitable vehicle (e.g. sterile, pyrogen-free water, normal saline or 5% dextrose) before use. The pharmaceutical compositions containing peptides(s) may also contain other active ingredients such as antimicrobial agents, or preservatives, and in the case of pills, binders and fillers, potentially in timed-release form. The compositions may contain from 0.001-99% (w/v or, preferably, w/w) of the active material.

The compositions are administered in therapeutically or prophylactic effective does, i.e. 0.05-1000 mg of peptide per day, in particular 5-250 mg per day. Very large doses may be used as the peptide according to the invention is non-toxic. However, normally this is not required. The dose administered daily of course depends on the degree of inflammation and inflammatory response.

Administration by injection or infusion of the compositions for treatment of adults of approximately 70 kg of body weight, will often range from 2-250 mg of active material, which may be administered in the form of 1 to 4 doses over each day.

The invention will be useful in the prevention or treatment of illness or medical conditions, particularly those involving inflammation in the brain, such as in epilepsy, seizures, brain injury, stroke, spinal cord injuries, neuropathies, cognitive decline, Alzheimer's Disease, Parkinson's Disease, neuro-AIDS, dementia's, bipolar disease and depression, as well as other conditions with an underlying inflammatory pathogenesis, such as uveitis and macular degeneration in the eye, inflammatory diseases of the bowel, such as Crohn's Disease, ulcerative colitis, dysbiosis causing ‘leaky bowel’, as well as inflammatory diseases of the skin, such as psoriasis, rosacea, eczema, dermatitis, or periodontitis in the mouth, and the systemic inflammation associated with metabolic and endocrine disorders, particularly obesity, type 2 diabetes, cardiovascular disease and atherosclerosis.

The peptides of the invention may be used to manage the immune-related adverse events (irAEs) of so-called cancer “checkpoint inhibitors”, like an anti-CTLA-4 antibody or in immune reconstitution inflammatory syndromes as may occur with cessation of immunosuppressive therapies like natalizumab, a humanized monoclonal antibody against alpha-4 (α4) integrin.

In chronic inflammation a preponderance of energy expenditure pathways is switched on, leading to endocrine and hormonal changes such as insulin/IGF-1 resistance, hypoandrogenemia, hypovitaminosis D, mild hypercortisolemia, and increased activity of the sympathetic nervous system and the renin-angiotensin-aldosterone system which contribute to increased mortality.

All of these seemingly disparate conditions share activation of the specific innate immune pathways, as described herein, which may be modulated or suppressed by the subject inventions.

### The Invention Can Be Illustrated By The Following Non-limiting Examples

To test the hypothesis that all-D-peptides which retain receptor activity may be created, with utility in inflammatory conditions, such as may occur in brain after injury or elsewhere in the body, we first used molecular and cellular approaches to explore the inflammatory reaction in isolated immature human monocyte derived immature dendritic cells (iDCs). DCs are derived from differentiated immature monocytes and serve as the innate and adaptive antigen presenting cells of the liver, brain, skin, and other tissues. The brain DCs are called microglia and activation of these sentinel cells is an early host response to injury and pathogens, which triggers an inflammatory cascade.

To determine whether all-D-TTNYT (SEQ ID NO:1) blocks maturation of antigen-presenting dendritic cells, human PBMC's were isolated from peripheral blood by Ficoll-Paque centrifugation and then monocytes were isolated by negative selection using immunobeads (Miltenyi). Human monocyte derived immature dendritic cells (iDCs) were then generated by treating monocytes with GM-CSF/IL4.

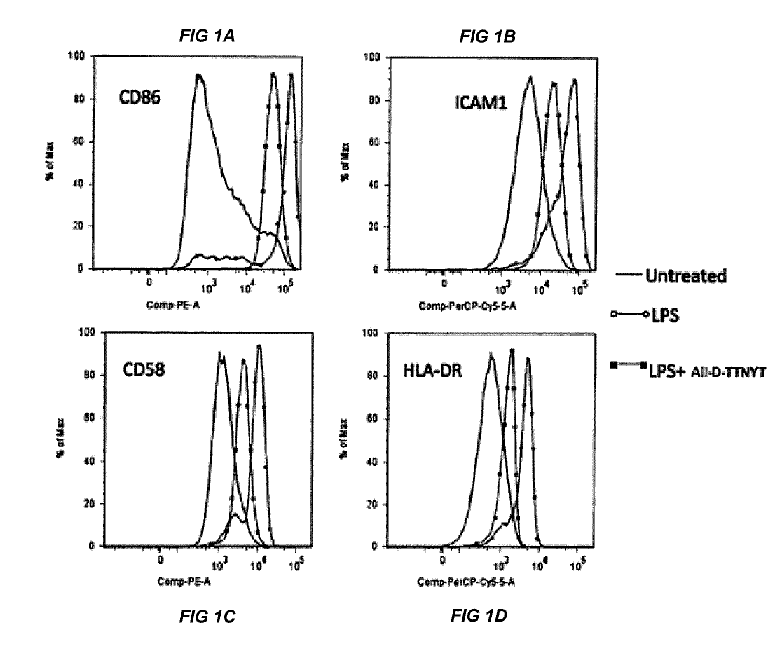

The iDCs were treated with all-D-TTNYT (SEQ ID NO:1), generic name RAP103 (12), at 10-12 M for 30 min. After 30 min LPS (100 ng/ml) was added to the cells and cells were analyzed after 48 hrs. for surface maturation markers using fluorescent labeled antibodies by flow cytometry. Shown in FIG. 1 are results of CD86, HLA-DR, CD58 (adhesion molecule) and ICAM1 (adhesion molecule) expression induced by TLR4/MyD88 activation (LPS), with and without, added all-D-TTNYT (SEQ ID NO:1).

In brain TLR4 is expressed by all parenchymal and non-parenchymal cell types, and contributes to tissue damage caused by a variety of etiologies. TLR4 activation leads to kinase activation (ERK1/2, p38, TBK1), transcription factor activation (NFκB, IRF3), and increased transcription of proinflammatory cytokines such as TNF-α, 1L-1β, and IL-6, and all of these pathways are implicated in epileptogenic and seizure activity (4, 9, 16, 37, 38). This immune signaling cascade is also thought to play a major role in neurodegeneration and other sequelae of brain injury that can be treated by the subject peptides.

As seen in the plots FIG. 1A-D, pretreatment of cells with all-D-TTNYT (SEQ ID NO:1) reduces expression of all the TLR4 stimulated maturation markers listed in the Figure. These surface molecules control T cell activation and localization in tissues. Microglia are the antigen-presenting DC's of the brain. Blockade of DC/microglial maturation and activation by TLR4/MyD88 would suppress inflammation in seizures, epilepsy, brain injury, and cognitive decline.

Our results showed that the maturation markers CD86, HLA-DR, CD58 (adhesion molecule) and ICAM1 (adhesion molecule) when stimulated via TLR4/MyD88 activation (LPS), are reduced by pre-treatment of the cells with all-D-TTNYT (SEQ ID NO:1). The expression of these maturation markers is well known in mediating immune cell trafficking and immune response in the context of tissue damage, neuronal toxicity, antigen recognition, microglial activation, as well as cytokine and chemokine release.

All-D-TTNYT (SEQ ID NO:1) however had no effect on DC maturation of these four markers caused by the antimicrobial peptide LL37, which binds to the insulin-like growth factor 1 receptor (IGF-1R) (not shown). The action of all-D-TTNYT (SEQ ID NO:1) therefore shows specificity for TLR4/MyD88.

TLR4 signaling occurs in cells of the brain including microglia, astrocytes, and even neurons, and such signaling mediates an inflammatory phenotype leading to neurotoxicity, SE, and seizure activity. Inhibiting DC maturation would have benefits in septic shock, which can also promote seizure activity, or other conditions with elevated TNF [symbol] levels. We conclude that all-D-TTNYT (SEQ ID NO:1) and related analogs can have a beneficial effect in seizures, epilepsy, brain injury, hyperexcitability, dyskinesias and cognitive decline, by modulating antigen presenting dendritic cell (microglia) activation in brain. In other dendritic cells, such as the liver Kuppfer cells or skin Langerhans cells, these peptides can reduce the inflammation underlying non-alcoholic steatohepatitis or psoriasis, to cite some examples.

FIG. 2 shows all-D[TTNYT (SEQ ID NO:1)], generic name RAP-103, is a dual-antagonist of CCR5 and CCR2 human monocyte chemotaxis. Monocytes were treated with the indicated doses of RAP-103 for 30 min before chemotaxis against human CCL2 (MCP-1) or CCL4 (MIP-1β) (both 50 ng/mL) for 90 min. Data (chemotactic index) are presented as mean ±SEM. The chemotactic index for MCP-1 without RAP-103 was 2.5-3.5 times over control, whereas for MIP-1 [symbol] without RAP-103, it was approximately 2 times over control. Data are presented as mean ±SEM. (*P<0.05, **P<0.01 vs RAP-103 untreated). Data are from (12), FIG. 1. The result shows a further useful action of these peptides as blocking of CCR2/CCR5 is beneficial in seizures, epilepsy, brain injury, and cognitive decline. Dual-chemokine CCR2/CCR5 receptor antagonists may have added therapeutic value by blocking multiple inflammatory pathways.

FIG. 3 shows three further examples of all-D-versions of DAPTA related pentapeptides, all-D-SSTYR (SEQ ID NO: 2), all-D-TTSYT (SEQ ID NO: 4), and all-D-NTSYR (SEQ ID NO: 7) are similarly antagonists of CCL2 human monocyte chemotaxis and would be expected to provide benefits in the inflammatory causes of seizures, epilepsy, brain injury, and cognitive decline. The methods employed are similar to those in FIG. 2.

FIG. 4 shows reductions of chemokines CCL2 and CCL3, the chemokine receptors CCR2 and CCR5, and the cytokines IL-1 and TNF [symbol] [symbol] in a rodent injury model of inflammation. The specific experimental details are provided in Padi, 2012 (12). Both Dala1-peptide T-amide and all-D-TTNYT (SEQ ID NO:1) share receptor targets, and biological effects indicating they are analogs that target the same pathological processes. All of the DAPTA related peptides that we describe are therefore expected to share the same actions, benefits, and therapeutic mechanisms, as is expected from structurally related analogs. The target biomolecules relevant to epilepsy, seizures, and brain injury are summarized in Table 1.

A further action of the subject peptides relevant to protecting against inflammation in epilepsy or seizures or brain injury in general, is the ability to decrease inflammatory cytokines, chemokines, and their receptors which underlie the disease processes. Table 1 illustrates that Dala1-peptide T-amide (DAPTA) lowers inflammatory cytokine levels in humans. The effect is shared by the pentapeptide all-D-TTNYT (SEQ ID NO:1) (RAP-103) which was administered by oral gavage, (0.05-1 mg/kg) for 7 days to nerve injured rats, who also showed reductions in key biomarkers identified in epilepsy, seizures, and brain injury.

TABLE 1
Summary of Biomarker Changes for DAPTA and
all-D-TTNYT (SEQ ID NO: 1)

| | | | | Phenotype |
|---|---|---|---|---|
| Biomarker | Species | Change | Compound | (M1 vs. M2) |
| IL-1 | Hu | decrease | DAPTA | M1 |
| IL-6 | Hu | decrease | DAPTA | M1 |
| IL-8 | Hu | decrease | DAPTA | M1 |
| IL-23 | Hu | decrease | DAPTA | M1 |
| TNFα | Hu | decrease | DAPTA | M1 |
| ICAM-1 | Hu | decrease | DAPTA | M1 |
| STAT3 | Hu | decrease | DAPTA | M1 |
| NFkB | Hu | decrease | DAPTA | M1 |
| TLR4/MyD88 | Rat | decrease | DAPTA | M1 |
| MCP-1 (CCL2) | Rat | decrease | all-D-TTNYT¹ | M1 |
| MIP-1α (CCL3) | Rat | decrease | all-D-TTNYT¹ | M1 |
| TNFα | Rat | decrease | all-D-TTNYT¹ | M1 |
| CCR2 | Rat | decrease | all-D-TTNYT¹ | M1 |
| CCR5 | Rat | decrease | all-D-TTNYT¹ | M1 |
| IL-1β | Rat | decrease | all-D-TTNYT¹ | M1 |
| IL-6 | Rat | decrease | all-D-TTNYT¹ | M1 |
| IL-4 | Hu | increase | DAPTA | M2 |
| IL-10 | Hu | increase | DAPTA | M2 |
| IL-13 | Hu | increase | DAPTA | M2 |

¹SEQ ID NO: 1

The predominant application of the invention is for control and prevention of seizures associated with brain injury, epilepsy or other central nervous system disorders, whose underlying causative pathogenesis relates to persistent inflammation via chemokine CCR2 and CCR5, TLR4, and cytokine pathways, all of which are attenuated by the subject peptides.

Macrophages can be activated to express several functional phenotypes, commonly referred to as classic and alternative activation, also termed M1 and M2, related to select biomarkers.

### REFERENCES

1. Dhote, F., A. Peinnequin, P. Carpentier, V. Bailie, C. Delacour, A. Foquin, G. Lallement, and F. Dorandeu. 2007. Prolonged inflammatory gene response following soman-induced seizures in mice. Toxicology 238: 166-176.
2. Dillman, J. F., C. S. Phillips, D. M. Kniffin, C. P. Tompkins, T. A. Hamilton, and R. K. Kan. 2009. Gene expression profiling of rat hippocampus following exposure to the acetylcholinesterase inhibitor soman. Chem Res Toxicol 22: 633-638.
3. Vezzani, A., J. French, T. Bartfai, and T. Z. Baram. 2011. The role of inflammation in epilepsy. Nat Rev Neurol 7: 31-40.
4. Uludag, I. F., T. Duksal, B. I. Tiftikcioglu, Y. Zorlu, F. Ozkaya, and G. Kirkali. 2015. IL-1β, IL-6 and IL1Ra levels in temporal lobe epilepsy. Seizure 26: 22-25.
5. Ravizza, T., B. Gagliardi, F. Noé, K. Boer, E. Aronica, and A. Vezzani. 2008. Innate and adaptive immunity during epileptogenesis and spontaneous seizures: evidence from experimental models and human temporal lobe epilepsy. Neurobiol Dis 29: 142-160.
6. Flannery, B. M., D. A. Bruun, D. J. Rowland, C. N. Banks, A. T. Austin, D. L. Kukis, Y. Li, B. D. Ford, D. J. Tancredi, J. L. Silverman, S. R. Cherry, and P. J. Lein. 2016. Persistent neuroinflammation and cognitive impairment in a rat model of acute diisopropylfluorophosphate intoxication. J Neuroinflammation 13: 267.
7. Zhao, Q. 2010. Dual targeting of CCR2 and CCR5 : therapeutic potential for immunologic and cardiovascular diseases. J Leukoc Biol
8. Shen, Y., H. Qin, J. Chen, L. Mou, Y. He, Y. Yan, H. Zhou, Y. Lv, Z. Chen, J. Wang, and Y. D. Zhou. 2016. Postnatal activation of TLR4 in astrocytes promotes excitatory synaptogenesis in hippocampal neurons. J Cell Biol 215: 719-734.
9. Cerri, C., S. Genovesi, M. Allegra, F. Pistillo, U. Püntener, A. Guglielmotti, V. H. Perry, Y. Bozzi, and M. Caleo. 2016. The Chemokine CCL2 Mediates the Seizure-enhancing Effects of Systemic Inflammation. J Neurosci 36: 3777-3788.
10. Louboutin, J. P., A. Chekmasova, E. Marusich, L. Agrawal, and D. S. Strayer. 2011. Role of CCR5 and its ligands in the control of vascular inflammation and leukocyte recruitment required for acute excitotoxic seizure induction and neural damage. FASEB J 25: 737-753.
11. Di Prisco, S., M. Summa, V. Chellakudam, P. I. Rossi, and A. Pittaluga. 2012. RANTES-mediated control of excitatory amino acid release in mouse spinal cord. J Neurochem 121: 428-437.
12. Padi, S. S., X. Q. Shi, Y. Q. Zhao, M. R. Ruff, N. Baichoo, C. B. Pert, and J. Zhang. 2012. Attenuation of rodent neuropathic pain by an orally active peptide, RAP-103, which potently blocks CCR2- and CCR5-mediated monocyte chemotaxis and inflammation. Pain 153: 95-106.
13. Victoria, E. C., E. C. de Brito Toscano, A. C. de Sousa Cardoso, D. G. da Silva, A. S. de Miranda, L. da Silva Barcelos, M. A. Sugimoto, L. P. Sousa, I. V. de Assis Lima, A. C. de Oliveira, F. Brant, F. S. Machado, M. M. Teixeira, A. L. Teixeira, and M. A. Rachid. 2017. Knockdown of C-C chemokine receptor 5 (CCR5) is protective against cerebral ischemia and reperfusion injury. Curr Neurovasc Res
14. Li, L., D. Zhi, Y. Shen, K. Liu, H. Li, and J. Chen. 2016. Effects of CC-chemokine receptor 5 on ROCK2 and P-MLC2 expression after focal cerebral ischaemia-reperfusion injury in rats. Brain Inj 30: 468-473.
15. Morganti, J. M., L. K. Riparip, A. Chou, S. Liu, N. Gupta, and S. Rosi. 2016. Age exacerbates the CCR2/5-mediated neuroinflammatory response to traumatic brain injury. J Neuroinflammation 13: 80.
16. Vitaliti, G., P. Pavone, F. Mahmood, G. Nunnari, and R. Falsaperla. 2014. Targeting inflammation as a therapeutic strategy for drug-resistant epilepsies: an update of new immune-modulating approaches. Hum Vaccin Immunother 10: 868-875.
17. Raychaudhuri, S. P., E. M. Farber, and S. K. Raychaudhuri. 1999. Immunomodulatory effects of peptide Ton Th 1/Th 2 cytokines. Intl Immunopharmacol 21: 609-15.
18. Ruff, M. R., M. Polianova, Q. E. Yang, G. S. Leoung, F. W. Ruscetti, and C. B. Pert. 2003. Update on D-ala-peptide T-amide (DAPTA): a viral entry inhibitor that blocks CCR5 chemokine receptors. Curr HIV Res 1: 51-67.
19. Rosi, S., C. B. Pert, M. R. Ruff, K. McGann-Gramling, and G. L. Wenk. 2005. Chemokine receptor 5 antagonist D-Ala-peptide T-amide reduces microglia and astrocyte activation within the hippocampus in a neuroinflammatory rat model of Alzheimer's disease. Neuroscience 134: 671-676.
20. Paoletti, I., V. De Gregorio, A. Baroni, M. A. Tufano, G. Donnarumma, and J. J. Perez. 2013. Amygdalin analogues inhibit IFN-gamma signalling and reduce the inflammatory response in human epidermal keratinocytes. Inflammation 36: 1316-1326.
21. Barrett, J. P., R. J. Henry, S. Villapol, B. A. Stoica, A. Kumar, M. P. Burns, A. I. Faden, and D. J. Loane. 2017. NOX2 deficiency alters macrophage phenotype through an IL-10/STAT3 dependent mechanism: implications for traumatic brain injury. J Neuroinflammation 14: 65.
22. Abboud, A., Q. Mi, A. Puccio, D. Okonkwo, M. Buliga, G. Constantine, and Y. Vodovotz. 2016. Inflammation Following Traumatic Brain Injury in Humans: Insights from Data-Driven and Mechanistic Models into Survival and Death. Front Pharmacol 7: 342.
23. Song, G. J., and K. Suk. 2017. Pharmacological Modulation of Functional Phenotypes of Microglia in Neurodegenerative Diseases. Front Aging Neurosci 9: 139.
24. Heseltine, P. N., K. Goodkin, J. H. Atkinson, B. Vitiello, J. Rochon, R. K. Heaton, E. M. Eaton, F. L. Wilkie, E. Sobel, S. J. Brown, D. Feaster, L. Schneider, W. L. Goldschmidts, and E. S. Stover. 1998. Randomized double-blind placebo-controlled trial of peptide T for HIV-associated cognitive impairment. Arch Neurol 55: 41-51.
25. Villemagne, V. L., R. L. Phillips, X. Liu, S. F. Gilson, R. F. Dannals, D. F. Wong, P. J. Harris, M. Ruff, C. Pert, P. Bridge, and E. D. London. 1996. Peptide T and glucose metabolism in AIDS dementia complex. J Nucl Med 37: 1177-1180.
26. Valekova, I., K. Jarkovska, E. Kotrcova, J. Bucci, Z. Ellederova, S. Juhas, J. Motlik, S. J. Gadher, and H. Kovarova. 2016. Revelation of the IFNα, IL-10, IL-8 and 1L-1β as promising biomarkers reflecting immuno-pathological mechanisms in porcine Huntington's disease model. J Neuroimmunol 293: 71-81.
27. Di Prisco, S., E. Merega, and A. Pittaluga. 2014. Functional adaptation of presynaptic chemokine receptors in EAE mouse central nervous system. Synapse 68: 529-535.
28. Mao, S. S., R. Hua, X. P. Zhao, X. Qin, Z. Q. Sun, Y. Zhang, Y. Q. Wu, M. X. Jia, J. L. Cao, and Y. M. Zhang. 2012. Exogenous administration of PACAP alleviates traumatic brain injury in rats through a mechanism involving the TLR4/MyD88/NF-κB pathway. J Neurotrauma 29: 1941-1959.
29. Saika, F., N. Kiguchi, Y. Kobayashi, Y. Fukazawa, and S. Kishioka. 2012. CC-chemokine ligand 4/macrophage inflammatory protein-lbeta participates in the induction of neuropathic pain after peripheral nerve injury. Eur J Pain 16: 1271-1280.
30. Lee, Y. K., D. Y. Choi, Y. Y. Jung, Y. W. Yun, B. J. Lee, S. B. Han, and J. T. Hong. 2013. Decreased pain responses of C—C chemokine receptor 5 knockout mice to chemical or inflammatory stimuli. Neuropharmacology 67: 57-65.
31. Ensign, L. M., R. Cone, and J. Hanes. 2014. Nanoparticle-based drug delivery to the vagina: A review. J Control Release
32. Hayball, P. J. 1996. Chirality and nonsteroidal anti-inflammatory drugs. Drugs 52 Suppl 5: 47-58.
33. Pert, C. B., J. M. Hill, M. R. Ruff, R. M. Berman, W. G. Robey, L. O. Arthur, F. W. Ruscetti, and W. L. Farrar. 1986. Octapeptides deduced from the neuropeptide receptor-like pattern of antigen T4 in brain potently inhibit human immunodeficiency virus receptor binding and T-cell infectivity. Proc Natl Acad Sci USA 83: 9254-8.
34. Brenneman, D. E., J. M. Buzy, M. R. Ruff, and C. B. Pert. 1988. Peptide T sequences prevent neuronal cell death produced by the envelope protein (gp120) of the human immunodeficiency virus. Drug Devel Res 15: 361-369.
35. Smith, C. C., P. L. Hallberg, P. Sacerdote, P. Williams, E. Sternberg, B. Martin, C. Pert, and M. R. Ruff. 1988. Tritiated Dala1-peptide T binding: A pharmacologic basis for the design of drugs which inhibit HIV receptor binding. Drug Devel Res 15: 371-379.
36. Stewart, J. M., and D. W. Woolley. 1965. All-D-bradykinin and the problem of peptide antimetabolites. Nature 206: 619-620.
37. Arisi, G. M., M. L. Foresti, K. Katki, and L. A. Shapiro. 2015. Increased CCL2, CCL3, CCL5, and 1L-1β cytokine concentration in piriform cortex, hippocampus, and neocortex after pilocarpine-induced seizures. J Neuroinflammation 12: 129.
38. Vezzani, A., and A. Friedman. 2011. Brain inflammation as a biomarker in epilepsy. Biomark Med 5: 607-614.

What is claimed is:

1: A method of treatment for epilepsy, seizures, or loss of brain function in a patient with a brain injury comprising the steps of:
preparing a composition comprising a D peptide and an acceptable carrier, said D peptide further comprises five contiguous amino acids having the general structure: A-B-C-D-E in which:
A is Ser, Thr, Asn, Glu, Ile.
B is Ser, Thr, Asp, Asn,
C is Thr, Ser, Asn, Arg, Trp,
D is Tyr, and
E is Thr, Ser, Arg, Gly.
wherein all amino acids are the D stereoisomeric configuration, and administering said composition to the patient in a therapeutically effective dose, wherein said composition acts to treat epilepsy, seizures, or loss of brain function in the patient.

2: The method as defined in claim 1 wherein said D peptide is TTNYT (SEQ ID NO: 1)

3: The method as defined in claim 1 further comprising, said D peptide is at most eight (8) all-D amino acid residues in length and contains five contiguous D amino acid residues that have a sequence selected from the group consisting of:
(SEQ ID NO: 1)
Thr Thr Asn Tyr Thr,
(SEQ ID NO: 2)
Ser Ser Thr Tyr Arg,
(SEQ ID NO: 3)
Ser Thr Asn Tyr Thr,
(SEQ ID NO: 4)
Thr Thr Ser Tyr Thr,
(SEQ ID NO: 5)
Asn Thr Ser Tyr Gly,
(SEQ ID NO: 6)
Glu Thr Trp Tyr Ser
(SEQ ID NO: 7)
Asn Thr Ser Tyr Arg
(SEQ ID NO: 8)
Ile Asn Asn Tyr Thr,
(SEQ ID NO: 9)
Ile Asp Asn Tyr Thr
(SEQ ID NO: 10)
Thr Asp Asn Tyr Thr
(SEQ ID NO: 11)
Thr Asp Ser Tyr Ser
(SEQ ID NO: 12)
Thr Asn Ser Tyr Arg,
and
(SEQ ID NO: 13)
Asn Thr Arg Tyr Arg.

4: The method as defined in claim 3 wherein said composition further comprises an oral pill having anti-inflammatory activity and wherein said peptide in said composition is present in a concentration having a range from 0.05 μg to 1000 μg.

5: The method as defined in claim 1 wherein E may be esterified, glycosylated, or amidated to enhance tissue distribution and entry into brain.

6: The method as defined in claim 1 wherein said composition further comprises an oral pill having anti-inflammatory activity and wherein said peptide in said composition is present in a concentration having a range from 0.05 μg to 1000 μg.

7: The method as defined in claim 6, wherein said anti-inflammatory activity consists of CCR5 or CCR2 receptor antagonism.

8: The method as defined in claim 6 wherein said anti-inflammatory activity consists of dual CCR5 and CCR2 antagonism.

9: The method of disease treatment as defined in claim 6 wherein said anti-inflammatory activity causes a reduction in inflammatory cytokines selected from: TNFα, IL-1, IL-6, IL-8, IL-12 and IL-23.

10: The method of disease treatment as defined in claim 6 wherein said anti-inflammatory activity causes an increase in anti-inflammatory cytokines, including IL-4 and IL-10.

11: The method of disease treatment as defined in claim 6 wherein said anti-inflammatory activity causes a reduction in the chemokines CLL2, CCL3 and the chemokine receptors CCR2 and CCR5.

12: The method as defined in claim 1 wherein said composition further comprises mannose and benzyl alcohol for preventing aggregation in liquid peptide solutions.

13: A method as defined in claim 1 wherein said composition further comprises Dala1-peptide T-amide (DAPTA) anti-inflammatory activity.

14: The method as defined in claim 13 wherein said composition further comprises mannose and benzyl alcohol for preventing aggregation in liquid peptide solutions.

15: A method as defined in claim 1 wherein said composition further comprises all-D-ASTTTNYT (SEQ ID NO:14) anti-inflammatory activity.

16: The method as defined in claim 15 wherein said composition further comprises mannose and benzyl alcohol for preventing aggregation in liquid peptide solutions.

17: A method as defined in claim 1 wherein said composition further comprises all-D-ASTTTNYT-NH₂ (SEQ ID NO:14) anti-inflammatory activity.


SEQUENCE LISTING (15 sequences)

SEQ ID NO: 1 - protein, 5 aa, Human immunodeficiency virus
TTNYT

SEQ ID NO: 2 - protein, 5 aa, Human immunodeficiency virus
SSTYR

SEQ ID NO: 3 - protein, 5 aa, Human immunodeficiency virus
STNYT

SEQ ID NO: 4 - protein, 5 aa, Human immunodeficiency virus
TTSYT

SEQ ID NO: 5 - protein, 5 aa, Human immunodeficiency virus
NTSYG

SEQ ID NO: 6 - protein, 5 aa, Human immunodeficiency virus
ETWYS

SEQ ID NO: 7 - protein, 5 aa, Human immunodeficiency virus
NTSYR

SEQ ID NO: 8 - protein, 5 aa, Human immunodeficiency virus
INNYT

SEQ ID NO: 9 - protein, 5 aa, Human immunodeficiency virus
IDNYT

SEQ ID NO: 10 - protein, 5 aa, Human immunodeficiency virus
TDNYT

SEQ ID NO: 11 - protein, 5 aa, Human immunodeficiency virus
TDSYS

SEQ ID NO: 12 - protein, 5 aa, Human immunodeficiency virus
TNSYR

SEQ ID NO: 13 - protein, 5 aa, Human immunodeficiency virus
NTRYR

SEQ ID NO: 14 - protein, 8 aa, Human immunodeficiency virus
ASTTTNYT

SEQ ID NO: 15 - protein, 6 aa, Human immunodeficiency virus
GTTNYT